(19) United States
(12) Patent Application Publication
(10) Pub. No.: US 2022/0142592 A1
(43) Pub. Date: May 12, 2022

(54) 3D LASER-ASSISTED POSITIONING SYSTEM

(71) Applicant: Amedo Smart Tracking Solutions GmbH, Bochum (DE)

(72) Inventor: Martin DELI, Mülheim a. d. Ruhr (DE)

(73) Assignee: Amedo Smart Tracking Solutions GmbH, Bochum (DE)

(21) Appl. No.: 17/601,101

(22) PCT Filed: Apr. 2, 2020
(86) PCT No.: PCT/EP2020/059410

§ 371 Date: Oct. 4, 2021

(30) Foreign Application Priority Data
Apr. 3, 2019 (DE) 10 2019 108 753.8

(51) Int. Cl. A61B 6/03; A61B 6/04

(52) U.S. Cl. CPC A61B 6/035; A61B 2090/374; A61B 6/0407

(57) ABSTRACT

Device (1) for positioning interventional instruments within an examination space, with a tomograph (11) for recording diagnostic image data of the examination space, a patient table (4) that can be moved transversely and/or rotated, at least one radiation unit (2, 2A, 2B, 2C) with a radiation source, which generates directed electromagnetic radiation, at least one carrier device (3, 3A, 3B, 3C) associated with the respective radiation unit (2, 2A, 2B, 2C), a control unit for orientation of the respective radiation unit (2, 2A, 2B, 2C) and of the patient table (4) according to the access and target points selected based on the diagnostic image data, wherein each radiation unit (2, 2A, 2B, 2C) is arranged movably with a first degree of freedom on the respective carrier device (3, 3A, 3B, 3C) and the carrier device (3, 3A, 3B, 3C) permits the mobility of this radiation unit (2, 2A, 2B, 2C) relative to the patient table (4), wherein an access point and the relative orientation of an instrument to reach a target point that lies in a trajectory of the instrument can be marked by means of the respective radiation unit (2, 2A, 2B, 2C), wherein the positioning of the carrier device (3, 3A, 3B, 3C) takes place in the coordinate system of the tomograph (11), to be precise without coordination with a second coordinate system of the carrier device (3, 3A, 3B, 3C) or the radiation source, wherein the access point and the target point can be selected independently of one another from the diagnostic image data, and the radiation unit (2, 2A, 2B, 2C) comprises an orientation device (6) for orientation of the radiation source and/or of the radiation (5) in at least a second and a third degree of freedom, wherein the second and the third degree of freedom relate to different movement axes.

The invention relates to a device for positioning instruments within an examination space, in which device a radiation element marks an access area and the relative orientation of the instrument to reach a target area by means of visible radiation.

The invention further relates to a method for positioning an instrument within an examination space and elements for use in the method.

In interventional radiology, puncture interventions such as biopsies, drainage, pain and tumor therapies, which are guided by sectional imaging techniques such as computer tomography (CT) or magnetic resonance imaging (MRI), for example, have now become firmly established. Their success is based both on a significant cost reduction compared with classic invasive operations and on intervention that is gentler for the patient.

MRI in particular is coming increasingly to the fore as an imaging procedure. No side effects of MRI are known to date and above all the patient is not exposed to any detrimental X-rays. At the same time, MRI permits free layer positioning, a highly detailed resolution of the soft tissue and a presentation of blood vessels free of contrast agents, making the use of this technique ideal for showing organs and possible pathologies of these.

One challenge in interventional radiology remains the correct placement of the instruments used for the minimally invasive intervention, such as needles, cannulas etc. This is because even if a suitable puncture point or a suitable access point can be marked relatively easily on the body surface, introduction of the instrument at the correct entry angle and with the correct penetration depth is still a major challenge for the operator.

Aids are now available that make it easier for the operator to introduce the instruments at the correct point and at a suitable angle. A device of this kind for positioning instruments is known from WO 2006125605 A1, for example, in which a needle positioning system according to the preamble of claim 1 is described. The known system comprises a radiation source, which on the basis of image data gathered by CT or MRI, for example, projects a visible beam onto the patient that displays the access point and entry angle selected according to image data. When the operator orients the invasive instrument within the trajectory, correct placement of the instrument is possible with regard to entry point and entry angle. What is also new in the known system is that it is installed in a fixed position and can therefore be used immediately. Other systems had to be laboriously positioned and registered prior to use, which made the exposure of a relatively extensive layer package necessary in the context of registration and was associated in CT with considerable radiation exposure for the patient. With the known system, on the other hand, the acquisition of a single layer is normally sufficient for intervention planning. In addition, time and costs are saved with this system, because due to the contactless mode of operation with a trajectory, sterile packaging of an otherwise necessary navigation apparatus is no longer required. The contactless mode of operation with a trajectory is also significantly safer than needle holders connected rigidly to the navigation system. Not least, the operator can freely select their puncture instruments in the known system. On account of the incorporation of the treatment table into the functionality of the navigation system, any number of needles, even those to be placed distant from one another, can be positioned without modifications to the system. A disadvantage of the known system, however, is that it is restricted to purely transverse needle guidance.

Although all other previously known systems also improve the precision of the instrument guidance, their clinical acceptance suffers due to the complex and time-intensive preparations. It is uncomfortable and prone to error, for example, if the entry point of the needle has to be determined conventionally in these systems. The laborious preparations, such as moving the system up, the sterile packaging and registration of the navigation system, for example, obstruct the clinical workflow. The rigid connection of the navigation system to the puncture needle positioned in the patient poses an additional safety risk for the patient. The tactile feedback and needle control options of the doctor are also limited by this. The limited choice of available needle holders not least limits the spectrum of operation instruments that can be deployed.

The narrow movement range of robotic navigation systems frequently leads in turn to the need to position, scan and register afresh, because the planned intervention lies outside of the set movement range of the system. If the needles in multi-needle interventions are not located close to one another, multiple positioning, scanning and registration is required.

Electromagnetic systems that are likewise available have a small range and the transmitter coil obstructs the puncture. The accuracy of such a system varies depending on metal parts in the environment of the puncture site. Expensive special needles are required for pertinent interventions and due to the narrow registration area only needles lying close to one another can be navigated. Special needles for special applications are scarcely obtainable.

In positioning using optical tracking systems, a reference frame must be connected immovably to the patient and the optical line between the cameras and the reference frame must be maintained. The reference frame connected to the puncture instrument obstructs the operator and must be supported in control exposures due to its weight.

Said limitations result in the majority of interventions still being carried out without navigation in the manner known as the “freehand technique”.

The object of the present invention is to develop a practicable navigation system that overcomes said limitations. Like the needle positioning system described in WO 2006125605 A1, the system must always be usable immediately without registration procedures, but its application may not be restricted to purely transverse accesses in the interventions. As well as transverse needle guidance, the navigation system according to the invention should accordingly also facilitate the free planning and display of caudocranially and craniocaudally angled needle paths, so that the medically or therapeutically most sensible access to the target location of the treatment or biopsy can always be selected. It is thereby prevented that an unfavorable access has to be used if necessary solely because only this lies in one of the planes that can be depicted by the system. A positioning system that can depict a flexible access path by means of a trajectory would be desirable, even if the access point and target point do not lie in the same transverse plane. The object of the invention, therefore, is to make a positioning system available that permits independent selection of the access and target point and correspondingly flexible orientation of the radiation for depicting the trajectory necessary for placement of the interventional instrument along the three spatial axes, thus the longitudinal axis, transverse axis and sagittal axis (also termed axes of movement below), and the trajectory is no longer limited in its representation to a transverse plane.

This object is achieved by an invention with the features of claim 1. Advantageous configurations each form the subject matter of the dependent claims. It should be pointed out that the features listed individually in the claims can also be combined with one another in any technologically sensible manner and thus demonstrate other configurations of the invention.

This object is achieved starting out from a device for positioning interventional instruments within an examination space according to the invention in that directed electromagnetic radiation marks an access area and the relative orientation of the instrument to reach the target area, which lies in the trajectory.

With the directed electromagnetic radiation emanating from a radiation source, the device according to the invention for positioning interventional instruments here permits the positionally accurate marking of a trajectory that corresponds to the extension of the straight line between the selected access area and the selected target area and in particular between the selected access point and the selected target point.

A beam or radiation in the range of radio frequencies, microwaves, infrared, also far or near infrared, of the UV range and in particular of the visible range are possible as directed electromagnetic radiation.

For accurate marking of the access area and the relative orientation of the instrument, it is necessary to use non-scattering, directed electromagnetic radiation. The use of laser light is therefore preferable, in particular of laser light in the visible range. Due to the application, a laser of low power is preferred, which is sufficient for marking the trajectory.

The device according to the invention for positioning interventional instruments comprises an imaging system, wherein the imaging system can be an MR, a CT, an ultrasonic system or another tomographic technique. Image sectioning procedures such as MRI and CT in particular are preferable. By recording the examination space, the diagnostic sectional imaging systems permit determination of the target area as well as of the access area, in particular the determination of the access point and target point. The trajectory extends the straight line between access area and target area and between access point and target point and thus directly defines the relative orientation of the instrument.

Furthermore, the inventive device comprises a patient table, which is movable and/or rotatable at least transversely. The material of the patient table and of any cushion placed thereon is expediently formed of MR-compatible material or material with low X-ray resistance.

To be able to orient the directed electromagnetic radiation according to the determined coordinates of a trajectory, at least one carrier device is associated with the device according to the invention. At least one radiation unit is associated with this at least one carrier device according to the invention, the at least one radiation unit comprising the radiation source itself or the radiation emanating from the radiation source, for example via a beam guide. For reasons of clarity, “carrier device” and “radiation unit” are referred to below in the singular, wherein devices according to the invention, as described, may also comprise several carrier devices and/or several radiation units.

A beam guide of this kind can comprise an arrangement of glass fibers. In the sense of this invention, the term “radiation” is to be understood as distinct from the term “radiation source” in such a way that radiation, unless explicitly mentioned otherwise, comprises the radiation conducted away from the radiation source in a radiation guide, thus in a glass fiber cable as described above, for example, in addition to the actual radiation. The radiation unit and thus also the corresponding radiation can be oriented by means of the carrier device in a first degree of freedom transversely relative to the patient table.

It is substantial for the functioning of the device according to the invention to establish a common examination space and thus a common coordinate system of both the imaging procedure and of the carrier device or of the radiation source associated with the radiation unit.

To execute a movement of the radiation relative to the patient table, the carrier device is preferably guided as a bridge in the form of a circle or of a segment of a circle on a radius around the patient table, so that the radiation unit with the radiation source is movable along the carrier device on a radius relative to the patient table.

In an alternative embodiment, more carrier devices with which at least one radiation unit is respectively associated can also be arranged around the patient table. One inventive embodiment is thus conceivable, for example, in which the device comprises three carrier devices and three radiation units, wherein precisely one radiation unit is associated with each carrier device. The carrier devices are arranged in a U-shape around the patient table with the opening of the “U” pointing downwards. The three carrier devices are accordingly arranged above and to the right and left of the patient table, preferably in a plane that lies perpendicular to the longitudinal axis of the patient table. The carrier devices in this embodiment are substantially straight, but can also be partly or wholly curved. The carrier devices can be separate from one another or connected to one another. To display the trajectory, the most suitable radiation unit is selected in each case, thus the radiation unit suitable for mapping the trajectory at the required angle to the object to be examined.

The carrier devices are preferably arranged at right angles to one another, but other angles are also conceivable.

This alternative embodiment with three carrier devices and three radiation units is advantageous, for example, if an arc-shaped carrier device is difficult to mount. Components that are formed substantially straight are also normally less susceptible to faults and easier to manufacture and are thus cheaper than curved parts.

It is possible for the expert to select other arrangements of carrier devices and radiation units according to the respective requirements.

The mobility of the radiation unit along the carrier device can be provided actively, thus by a drive located in the radiation unit, or passively, thus via a drive located outside of the radiation unit. A passive drive outside of the radiation unit is preferable. A passive drive of this kind can be realized, for example, by fixing the radiation unit on a cable- or belt-shaped transport element, which is provided movably along the carrier device. The transport element can be moved, for example, by a stepper motor of the type described below and the radiation unit thus brought into a desired position along the carrier device. The carrier device is accordingly designed preferably to take up such a transport element with at least one drive motor.

The carrier device comprises a power supply for the radiation unit. Such a power supply can be provided in the form of contact rails, for example. It is advantageous with contact rails that no cabling could impede the freedom of movement of the radiation unit. The radiation unit accordingly has contacts for receiving the power from the contact rail or rails.

To enable other degrees of freedom in the relative orientation of the radiation with respect to the patient or the patient table, the radiation unit comprises an orientation device according to the invention. The orientation device facilitates the orientation of the radiation in at least two other degrees of freedom, thus at least in a second and a third degree of freedom. Due to the realization of at least three degrees of freedom independent of one another, an orientation of the radiation starting out from the radiation source is made possible according to the invention in combination with the movement options of the patient table of the respective imaging apparatus at virtually any angle and at virtually any point on the surface of the object to be treated.

Insofar as reference is made to axes and to planes to describe the present invention, it is obvious to the expert that, on account of the common coordinate system according to the invention, both the corresponding spatial axes and spatial planes and the corresponding body axes (or movement axes) and body planes are always meant.

As described above, the first degree of freedom permits the orientation of the entire radiation unit transversely (within a transverse plane) relative to the patient table. The second degree of freedom permits additional orientation of the radiation within this transverse plane. The first and second degree of freedom thus enable orientation of the radiation within a transverse plane along the sagittal and transverse axis holding this plane. The third degree of freedom permits additional orientation of the radiation out of the transverse plane along the longitudinal axis of the patient or of the patient table. The radiation is accordingly able to be oriented according to the invention with three degrees of freedom along the three axes of movement.

To implement the orientation of the radiation, the orientation device comprises orientation elements, in particular articulations and mirror elements, the mirror elements being provided movably. For the mobility desired in each case, the expert selects suitable joints as articulations, for example pivot joints, ball joints etc. The orientation elements for orienting the radiation preferably comprise at least one pivot joint and at least one mirror element. Embodiments are also conceivable, however, in which the orientation elements comprise only articulations or only mirror elements.

The radiation can preferably be pivoted by the orientation elements of the orientation device along the longitudinal axis and the transverse axis respectively by up to 100°, preferably by up to 90° and in particular by up to 88°. Pivoting of 90° along the longitudinal axis signifies, for example, that starting from a vertical beam onto the patient table, pivoting of the radiation by 45° in a first direction along the longitudinal axis can take place, for example to the head end of the patient table, and pivoting of the radiation by 45° in a second direction along the longitudinal axis can take place, for example to the foot end of the patient table. The same applies to pivoting along the transverse axis.

Every trajectory required for placement of an instrument can be reproduced in this way in the coordinate system of the examination space and thus also on the patient. A goniometer can be used for precise adjustment of the laser beam relative to the patient table in the coordinate system of the examination space. For the preferably automated movement of the articulations and mirror elements for orientation of the radiation, the use of stepper motors capable of covering very small angular ranges of up to 10⁻⁴° is preferred.

To transmit the data to the radiation unit for controlling the motors and corresponding orientation of the orientation elements, other contact rails for data transmission can be provided in the carrier device in addition to the contact rails for the power supply. The data transfer to the radiation unit can naturally also take place in another way, in particular via cable or also wirelessly. The expert selects the suitable option here.

In coordination with the computer program of the inventive device, the orientation of the patient table can be carried out likewise in small increments purely transversely up/down, forwards/backwards or to the right/left, for example. A slight inclination or rotation of the patient table can likewise take place, but should only take place to a small extent in order to avoid a change in the patient's own position in the examination space and thus in the coordinate system of the device.

As already stated, the existing coordinate system of an imaging procedure such as of a computer tomograph, a magnetic resonance tomograph or another imaging system is used according to the invention for the control and orientation of electromagnetic radiation within the examination space.

The computer program required for this can be implemented on existing hardware of the imaging systems, for example. The computer program enables control of the positioning device according to the invention.

From volume image data the computer program calculates possible position data for access areas and target areas, preferably access points and target points, or access areas and target areas, preferably access points and target points, can be selected by the operator on the basis of the image data. Based on the selection, the program calculates the trajectory required for the correct positioning and via a control unit it controls a suitable carrier device with an associated radiation unit, so that the directed electromagnetic beam lies in the calculated trajectory.

Compared with the prior art, the device according to the invention has the advantage that it can display the possible freely angled access paths of invasive interventions, such as biopsies, drainage, drug deliveries or tumor treatments contactlessly. The operator is not limited to transverse access but can select the best access path possible with the device now proposed.

The invention and the technical environment are explained in greater detail below based on the figures. It should be pointed out that the figures show a particularly preferable implementation variant of the invention. The invention is not limited to the implementation variant shown, however. In particular, the invention comprises, as far as technically feasible, any combinations of the technical features detailed in the claims or described as relevant to the invention in the description.

The figures show:

FIG. 1 the arrangement of the inventive device on a CT

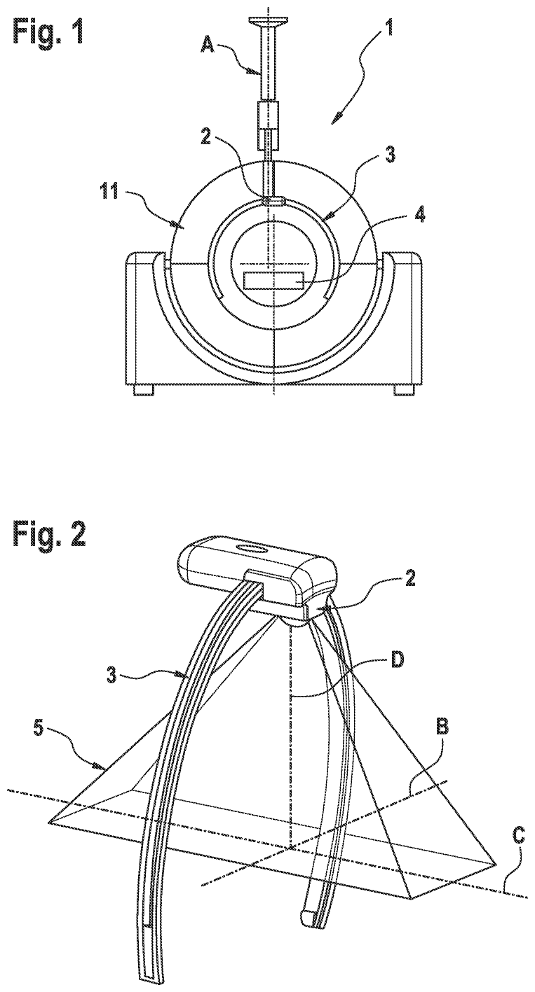

FIG. 2 a schematic overview of the inventive device with a depiction of the possible angles of incidence of the radiation

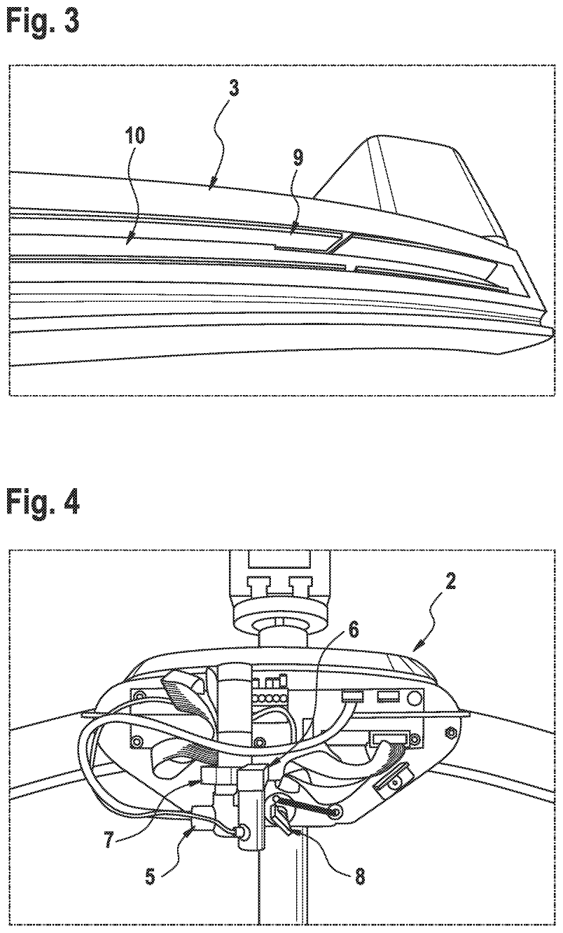

FIG. 3 a detailed view of the carrier device with contact rails and transport element

FIG. 4 a detailed view of the radiation unit with radiation guide and orientation elements

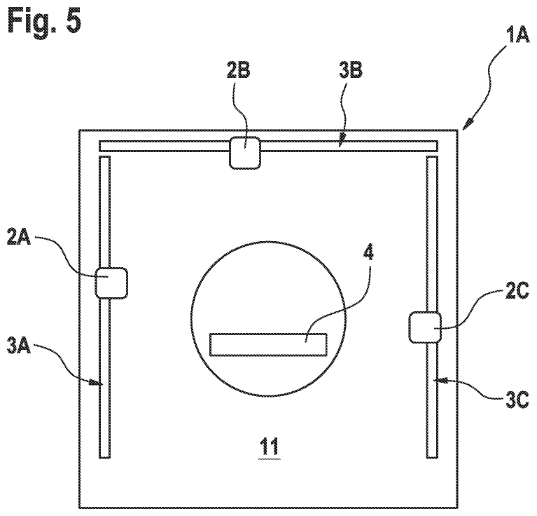

FIG. 5 a schematic view of an alternative embodiment of the device with three radiation units and three carrier devices

FIG. 1 shows the inventive device 1 in this case in combination with a CT as an imaging system 11. The inventive device 1 further comprises the radiation unit 2 and the carrier device 3. The carrier device 3 is arranged in a circular segment around the patient table 4. The radiation unit 2 is provided movably on the carrier device 3. The carrier device 3 is connected via a holding element A to a defined position in relation to the tomograph 11, for example to the ceiling of the CT room or to the tomograph 11.

FIG. 2 shows a part of the inventive device 1, namely the radiation unit 2 and the carrier device 3. According to the invention, the radiation 5 emanating from the radiation unit 2 can be oriented both along the transverse axis B and the longitudinal axis C. This is one of the substantial advantages of the inventive device 1, as it thus permits the selection of an access path also outside of a purely transverse orientation within the transverse plane.

The radiation 5 emanating from the radiation unit 2 is preferably pivotable by the orientation elements 7, 8 of the orientation device 6 (not depicted) along the transverse axis B and the longitudinal axis C respectively by up to 90°. Pivoting of 90° along the longitudinal axis signifies, for example, that starting out from an imaginary vertical beam D onto the patient table (not shown), pivoting of the radiation by 45° in a first direction along the longitudinal axis C can take place, for example to the head end of the patient table, and pivoting of the radiation by 45° in a second direction along the longitudinal axis C can take place, for example to the foot end of the patient table. The same applies to pivoting along the transverse axis B.

FIG. 3 shows a detailed view of the carrier device 3 with contact rails 9 and transport element 10. The contact rails 9 serve to supply power to the radiation unit 2, which has corresponding contacts (neither shown). These or other contact rails 9 can also be provided for transmitting data to the radiation unit 2. The transport elements 10 are provided here as belts, which can be driven via motors (not shown). The radiation unit 2 is connected to the transport element 10 in this case and can be oriented via this as a passive drive along the carrier device 3.

FIG. 4 shows a detailed view of the open radiation unit 2 with orientation device 6. For implementing the orientation of the beam 5, the orientation device 6 comprises orientation elements 7, 8, here a pivot joint 7 for orientation of the beam guide 5 and a mirror element 8 for orientation of the radiation 5 emanating from the beam guide. The orientation elements 7, 8 are provided movably via motors. The expert selects the joints 7 according to the mobility desired in each case. Embodiments are thus also conceivable with ball joints etc., for example. The expert likewise selects the mirror elements 8 according to his expert knowledge.

In addition to the first degree of freedom possible via the mobility of the radiation unit 2 along the carrier device, the orientation device 6 enables the radiation 5 to be oriented by means of the orientation elements 7, 8 in at least two other degrees of freedom, thus at least in a second and a third degree of freedom. It is important in this case that the three degrees of freedom at least partially relate to different axes, as shown in FIG. 2.

FIG. 5 shows an alternative embodiment of the device 1A according to the invention with three radiation units 2A, 2B, 2C, each of which is associated with a carrier device 3A, 3B, 3C. The technical construction of the radiation units 2A, 2B, 2C and the carrier devices 3A, 3B, 3C corresponds to the construction described previously. This alternative embodiment of the device 1A differs from the embodiment described previously with just one radiation unit and a carrier element in the form of a circular arc (as shown in FIG. 2) in that here it is not a single circular-arc carrier device with just one radiation unit arranged movably thereon that is provided. Here three radiation units 2A, 2B, 2C are provided, which are each associated with a carrier device 3A, 3B, 3C. The carrier devices 3A, 3B, 3C are preferably provided formed straight, but embodiments are also conceivable in which all or individual carrier devices 3A, 3B, 3C can be curved.

The carrier devices 3A, 3B, 3C are preferably arranged in a “U” shape around the patient table 4 such that the opening of the “U” points downwards. The carrier devices 3A, 3B, 3C are arranged here preferably at right angles to one another, arrangements at another angle also being conceivable.

### LIST OF REFERENCE CHARACTERS

1 Device
2 Radiation unit
3 Carrier device
4 Patient table
5 Radiation
6 Orientation device
7 Orientation element, articulation
8 Orientation element, mirror
9 Contact rail
10 Transport element
11 Tomograph
A Holding element
B Transverse axis
C Longitudinal axis
D Vertical beam (sagittal axis)

1. Device for positioning interventional instruments within an examination space, with
a tomograph for recording diagnostic image data of the examination space,
a patient table that can be moved transversely and/or rotated,
at least one radiation unit with a radiation source, which generates directed electromagnetic radiation,
at least one carrier device associated with the respective radiation unit,
a control unit for orientation of the respective radiation unit and of the patient table according to the access and target points selected based on the diagnostic image data,
wherein each radiation unit is arranged movably with a first degree of freedom on the carrier device associated with it and the carrier device permits the mobility of the respective radiation unit relative to the patient table,
wherein an access point and the relative orientation of an instrument to reach a target point that lies in a trajectory of the instrument can be marked by means of the respective radiation unit,
wherein the positioning of the carrier device takes place in the coordinate system of the tomograph, to be precise without coordination with a second coordinate system of the respective carrier device or the respective radiation unit,
wherein the access point and the target point can be selected independently of one another from the diagnostic image data and the respective radiation unit comprises an orientation device for orientation of the radiation source and/or of the radiation in at least a second and a third degree of freedom, wherein the second and the third degree of freedom relate to different movement axes.

2. Device according to claim 1, wherein the movement axes of the second and third degree of freedom correspond to the transverse axis and the longitudinal axis of the patient table.

3. Device according to claim 1, wherein the orientation device for orientation of the radiation comprises orientation elements, in particular at least one pivot joint and/or at least one mirror element.

4. Device according to claim 3, wherein of the orientation elements, a pivot joint is associated with the orientation of the beam guide and a mirror element is associated with the orientation of the radiation.

5. Device according to claim 1, wherein the radiation is pivotable by the orientation elements of the orientation device along the longitudinal axis and the transverse axis respectively by 100°, preferably by 90° and in particular by 88°.

6. Device according to claim 1, wherein the tomograph is a magnetic resonance imaging system (MRI) or a computer tomograph (CT).

7. Device according to claim 1, wherein the diagnostic image data from which the access point and the target point can be selected comprises several image layers.

8. Device according to claim 1, wherein the carrier device comprises a power supply for the radiation unit.

9. Device according to claim 1, wherein the carrier device comprises a drive for moving the radiation unit.

10. Device according to claim 1, wherein the device comprises precisely one radiation unit and precisely one carrier device, wherein the one carrier device permits the mobility of the one radiation unit along a radius relative to the patient table.

11. Device according to claim 1, wherein the device comprises three radiation units and three carrier devices, wherein a radiation unit is associated with each of the three carrier devices and wherein the carrier devices permit the mobility of the respective radiation unit along a path relative to the patient table and wherein the three carrier devices are arranged substantially in a “U” shape around the patient table.
